(19) United States
(12) Patent Application Publication
(10) Pub. No.: US 2025/0040813 A1
(43) Pub. Date: Feb. 6, 2025

(54) VASCULAR RESISTANCE AND BLOOD PRESSURE MEASUREMENT USING COMBINED PIEZOELECTRIC AND PHOTOPLETHYSMOGRAM MEASUREMENT

(71) Applicant: REGENTS OF THE UNIVERSITY OF MICHIGAN, Ann Arbor, MI (US)

(72) Inventors: Kevin R. Ward, Superior Township, MI (US); Sardar Ansari, Ypsilanti, MI (US); Kenn Oldham, Ann Arbor, MI (US); Lu Wang, Ann Arbor, MI (US)

(21) Appl. No.: 18/717,359

(22) PCT Filed: Dec. 8, 2022
(86) PCT No.: PCT/US2022/052280

§ 371 Date: Jun. 6, 2024

Related U.S. Application Data
(60) Provisional application No. 63/287,273, filed on Dec. 8, 2021.

(51) Int. Cl. A61B 5/021; A61B 5/00; A61B 5/02; A61B 5/022; A61B 90/00

(52) U.S. Cl. CPC A61B 5/02116; A61B 5/02007; A61B 5/02028; A61B 5/02141; A61B 5/02241; A61B 5/6826; A61B 5/6831; A61B 5/7242; A61B 5/7246; A61B 5/7271; A61B 90/06; A61B 5/6819; A61B 5/6822; A61B 5/6823; A61B 5/6824; A61B 5/6829; A61B 2090/064; A61B 2560/0462; A61B 2562/0247; A61B 2562/0266

(57) ABSTRACT

Systems and methods are provided for non-invasively measuring one or more hemodynamic variables using a peripheral arterial region wearable device having a strain sensor and a photoplethysmogram sensor. Different line fit profiles are determined from device collected signals indicative of the strain response versus externally applied cuff pressure and the blood volume and/or the blood circulation changes versus externally applied cuff pressure. From these line fit profiles, external cuff pressure affected line fit features are determined, from which hemodynamic variables for the peripheral arterial region such as blood pressure and/or a systemic vascular resistance are then determined.

### CROSS-REFERENCE TO RELATED APPLICATIONS

This application claims the benefit of U.S. Provisional Patent Application No. 63/287,273, filed Dec. 8, 2021. The entire disclosure of which is incorporated herein by reference in its entirety.

### STATEMENT OF GOVERNMENT SUPPORT

This invention was made with government support under CMMI1562254 awarded by the National Science Foundation. The government has certain rights in the invention.

### FIELD OF THE DISCLOSURE

The present disclosure relates to cardiovascular monitoring and, more particularly, to techniques for determining hemodynamic variables from wearable devices under blood pressure cuff induced conditions.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Hemodynamic variables such as blood pressure (BP) and vascular resistance (VR) are key physiological indicators of health. They reflect the overall physical wellbeing of an individual. For example, hypertension (i.e., high BP) is the gateway disease for more serious medical conditions such as diabetes, ischemic heart disease, heart failure, stroke, and chronic kidney disease, to name a few.

Despite the importance of BP as a major health indicator in a large range of medical conditions, current clinical practice use century-old techniques (auscultatory and oscillometric methods) to measure BP. There is an ongoing need for more versatile BP monitors that can be incorporated into smart wearable devices to track BP accurately and continuously without interfering with daily physical activity. While this need is widely recognized, numerous of the proposed solutions suffer from shortcomings.

The limitations of conventional measurement techniques affect vascular resistance measurements, as well. Peripheral arterial monitoring is used to detect changes in vascular resistance (VR). Changes in VR is one of the body's primary compensatory mechanisms in response to cardiovascular stress, largely through contraction or dilation of peripheral arteries. However, changes in peripheral arterial radius are difficult to monitor continuously; direct measurement is possible only through image-based methods and these are unavailable on a continuous basis. Other measurement techniques have been proposed based on metrics such as pulse transit time (PTT) or waveform analysis of the photoplethysmogram (PPG), but they have limited accuracy.

The present inventors have previously proposed techniques for non-invasive methods for tracking local peripheral artery behavior using a wearable device having a combination of pressure and optical sensing. Arterial pressure fluctuations are transmitted to a piezoelectric polymer (poylvinylidene difluoride (PVDF)) ring and a photoplethysmogram (PPG) analyzer, i.e., a PPG. Together with a model for intervening tissue, these techniques monitored relative changes in artery radius, albeit without providing an absolute measure of arterial status. Even the vascular resistance changes inferred from estimated arterial radius variation tracked closely with systemic VR measurements taken by gold standard cardiovascular catheterization in test subjects, showing the effectiveness of such conventional techniques.

Arterial dynamics of the cardiovascular system have been studied by various other researchers, as well. As an example, a model relating pulse transit time and blood pressure to changes in arterial dynamics was developed by Mukkamala, et al. Other researchers have used linear or nonlinear constitutive equations to describe the pressure/cross-sectional area relationship of peripheral arteries, with more complex models also available. Sherwin et al., for example, included the effect of vessel tapering by considering a varying initial cross-sectional area of the vessel. In other examples, vessel collapse has been modelled by adapting the vessel properties and considering pressure changes in the collapsed vessel area. Yet, still, other researchers have studied the pulse wave transmission in collapsible vessels. For example, Elad et al. studied the unsteady fluid flow through collapsible tubes.

Unfortunately, many conventional cardiovascular model techniques use one dimensional (1D) cardiovascular models, which assume only uniform vessel properties. For example, although the non-linear effects, such as the vessel tapering and curvature, viscoelastic properties, and inertia of the vessel, etc., have been analyzed previously, these were not universally considered in 1D cardiovascular models. In response, some have proposed multi-scale modelling techniques, in which zero dimension (0D) models are coupled with 1D, two dimensional (2D) and/or three dimensional (3D) models to form complete representations of the cardiovascular system. Pontrelli coupled a 1D arterial pulse wave transmission model to two 0D compartment models, for example. In another example, Migliavacca et al. embedded a 3D model of a systemic to pulmonary conduit in a 0D multiple branched circulation system model. In yet another example, Formaggia et al. coupled a 1D model to a 3D model to reduce computational complexity in a hemodynamic system. These models illustrate the importance of both longitudinal and radial behavior in interpreting arterial motion. Yet, none of these models provide sufficiently accurate assessments of hemodynamic variables of a subject, especially for variables related to arterial conditions.

There is a need for effective solutions for continuous, accurate, and portable measurement of hemodynamic variables to properly monitor and diagnose diseases in patients.

### SUMMARY

Peripheral artery status is a key hemodynamic variable that provides a physiological indicator of the body's cardiovascular response to both acute and chronic medical conditions. Conventional techniques for measuring peripheral artery status have been shown to lack accuracy and lack an ability to be performed quickly or continuously.

The present application, by contrast, describes systems and methods for determining peripheral artery behavior non-invasively by combining measurements from two different sensors, each employing a different measuring modality. The two sensors, for example, may be a photoplethysmograph (PPG) sensor and a strain sensor, such as a piezoelectric (e.g., polyvinylidene difluoride (PVDF)), applied to peripheral artery. More particularly, these sensors are operated while applying an outside pressure-varying cuff to the subject. The systems and methods herein may include a mechanical model for the local artery and tissue responses that is developed to receive output signals from the these sensors, taking during changes in cuff pressure, and generate hemodynamic variables indicating a health of a subject. In various implementations, the mechanical model capture time-dependent features and pressure-dependent features present in the PPG and PVDF signals as a function of applied cuff pressures. The mechanical models themselves may include a number of different features assessed during maneuvers applied to subjects to perturb blood pressure (BP) and vascular resistance (VR) in response to changes in cuff pressures as a method of generating the mechanical models. From the trained models, in various implementations, the system and methods are able to generate hemodynamic variables, including more accurate non-invasive blood pressure (BP) measurements and, for the first time, systemic vascular resistance (SVR) by using a multiple sensor device operating under applied cuff pressure.

In accordance with an example, a device for non-invasively measuring one or more hemodynamic variables of a subject, the device comprising: a strain sensor positioned in the device to generate signals indicative of strain response at a peripheral arterial region of the subject; a photoplethysmogram sensor positioned in the device to generate signals indicative of a blood volume and/or blood circulation changes at the peripheral arterial region; a support structure physically coupled to the strain sensor and the photoplethysmogram sensor to physically support the device at the peripheral arterial region; and a processor in communication with the strain sensor and the photoplethysmogram sensor and configured to: collect the signals indicative of the strain response and the signals indicative of the blood volume and/or blood circulation changes during changes in an externally applied cuff pressure to the subject; determine a first line fit profile of the signals indicative of the strain response versus externally applied cuff pressure; determine a second line fit profile of the signals indicative of the blood volume and/or blood circulation changes versus externally applied cuff pressure; and determine external cuff pressure affected line fit features from the first line fit and from the second line fit and from the external cuff pressure affected line fit features determining a blood pressure and/or a systemic vascular resistance for the peripheral arterial region, as the one or more hemodynamic variables.

In accordance with another example, a method for noninvasive measurement of one or more hemodynamic variables of a subject, the method comprising: using a strain sensor, collecting signals indicative of strain response at a peripheral arterial region of the subject during changes in an externally applied cuff pressure to the subject; using a photoplethysmogram sensor, collecting signals indicative of a blood volume and/or blood circulation changes at the peripheral arterial region during the changes in the externally applied cuff pressure to the subject; determining, in a processor, a first line fit profile of the signals indicative of the strain response versus externally applied cuff pressure; determine, in the processor, a second line fit profile of the signals indicative of the blood volume and/or blood circulation changes versus externally applied cuff pressure; and determine, in the processor, external cuff pressure affected line fit features from the first line fit and from the second line fit and form the external cuff pressure affected line fit features determining a blood pressure and/or a systemic vascular resistance for the peripheral arterial region, as the one or more hemodynamic variables.

### BRIEF DESCRIPTION OF THE DRAWINGS

FIGS. 1A, 1B, and 1C are diagrams of example multi-modal sensor devices and cuff device placed over peripheral arterial regions for tissue and sensor model development, in accordance with an example. FIG. 1D is a diagram of an example control circuit layout for a multi-modal sensor device, in accordance with an example.

FIG. 2 is a side view of a schematic of an example swine fore-limb under pressure cuff, in cross section view along the artery, showing model variables, in accordance with an example.

FIG. 3 is a plot of mean arterial pressure (MAP, in blue) and systemic vascular resistance (SVR, in yellow) perturbed by norepinephrine (NE) infusions and other maneuvers, while cuff inflations introduce intermittent external cuff pressure (P_{cuff}, in red) over the course of a sample swine experiment, in accordance with an example.

FIG. 4 is a plot of external pressure (P_{cuff}, in red) drops from maximum cuff inflation, showing that cardiac waveforms reappear from their fully and largely suppressed state for both a PVDF sensor and a PPG sensor (U_{pdvf}, in blue and U_{ppg}, in green, respectively), reaching maximum amplitudes at different rates and cuff pressures, in accordance with an example.

FIG. 5 is a plot of sensor signal amplitudes ((U_{pdvf}, blue and U_{ppg}, green) versus external cuff pressure for a single cuff deflation cycle (rolling average)), where red dash lines are systolic and diastolic blood pressures, in accordance with an example.

FIGS. 6A and 6B are plots, respectively, showing sample experimental PPG (FIG. 6A) and PVDF (FIG. 6B) sensor signals amplitude (z-axis) and cuff pressure (x-axis) with increasing SVR (y-axis), in accordance with an example. The color indicated different level of SVR form low (red) to high (turquoise).

FIG. 7 is a plot of various multiple linear regression (MLR) model parameters from the sample signal profile (line fit profiles), in accordance with an example.

FIG. 8 illustrates plots of experimental (dots) and modeled (solid lines) of PPG sensor signals amplitude (z-axis) and cuff pressure (x-axis) with increasing SVR (y-axis), in accordance with an example. The color indicated different level of SVR from low (red) to high (turquoise).

FIG. 9 illustrates plots of experimental (dots) and modeled (solid lines) PVDF sensor signals amplitude (z-axis) and cuff pressure (x-axis) with increasing SVR (y-axis), in accordance with an example. The color indicated different level of SVR from low (red) to high (turquoise).

FIG. 10 is a plot of non-invasive SVR estimation versus invasive SVR measurement; training and testing data, in accordance with an example, with R²=0.83.

FIGS. 11A-11C illustrates plots of estimated systolic pressure versus invasive systolic pressure for the three swine subjects using an initial method of single feature detection during cuff pressure during deflation (P̅_{sys}) and best fit to the proposed model during cuff deflation (\hat{P}_{sys}), in accordance with an example.

FIGS. 12A-12C illustrates plots of estimated diastolic pressure versus invasive diastolic pressure for the three swine subjects using a simple method of single feature detection during cuff pressure during deflation (P̅_{dia}) and best fit to the proposed model during cuff deflation (\hat{P}_{dia}).

FIG. 13 is a block diagram of an example signal processing device to determine hemodynamic variables such as BP and SVR from measured pulse signals using the models developed herein, in accordance with an example herein.

FIG. 14 illustrates a method for noninvasive measurement of hemodynamic variables of a subject using the example signal processing device of FIG. 13, in accordance with an example herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DESCRIPTION

The present application describes systems and methods for vascular resistance and blood pressure measurement using combined strain and photoplethysmogram (PPG) measurements using wearable devices having multiple sensors therein. In various implementations, these wearable devices, and methods of operation thereof, include one or more models developed to account for features of interaction between the underlying tissue and the sensors embedded in the wearable device and to generate hemodynamic variables such as BP and/or SVR. In various implementations, the wearable devices include multiple sensors to increase effectiveness of the models, such as piezoelectric (PVDF) strain sensors and PPG sensors. In various implementations, for example, the wearable device is a ring device or ring-like device that provides non-invasive peripheral artery monitoring, while outside pressure is manipulated by a pressurized cuff.

In various implementations, the systems and methods include models that incorporate limited longitudinal behavior with peripheral arterial contraction dynamics beneath a two-sensor (PVDF/PPG sensor) assembly to interpret variations in sensor signals under varying external pressures. These wearable devices may include models designed to include analysis of differences between the different sensor responses to changes in external pressure. Moreover, the wearable devices may include models that include improved estimates of physiological parameters such as SVR and blood pressure over a larger number of subjects. Moreover still, in various implementations, the systems and methods include a 2D model for arterial blood pressure (BP), volumetric flow rate, and artery radius with varying outside cuff pressures, and the 2D model can be used to determine hemodynamic variables such as SVR.

In an example implementation, we describe an experiment performed on swine test subjects from which peripheral artery and local tissue data were collected. We compared experimental and modeled output from which the changing amplitude of sensor signals was shown to strongly correlate with invasively measured systemic vascular resistance (SVR) data. A multiple linear regression model was proposed to estimate SVR using the selected features modeled from the PVDF signal behavior and from the PPG signal behavior. A 2D model allowed for refinement of non-invasive blood pressure measurements with information collected while varying cuff pressure to the subject. In various implementations, a model is proposed to account for contrasting effects in non-invasive pressure-based measurements versus optical measurements of peripheral artery behavior under varying external pressure.

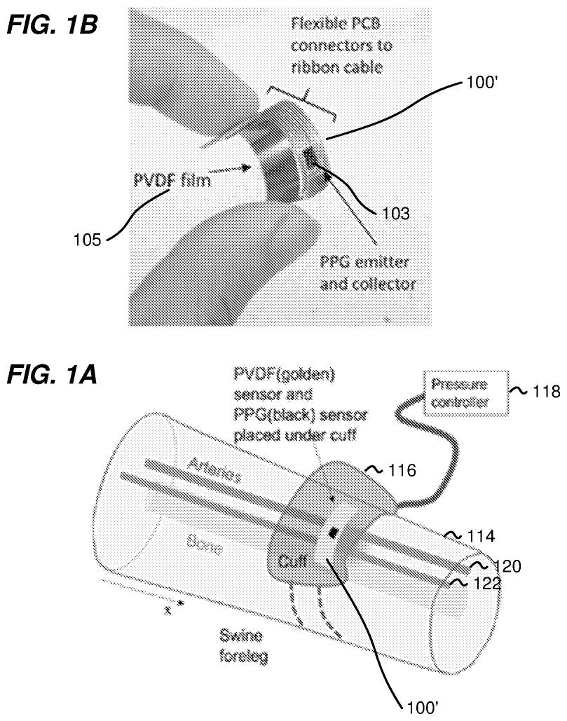

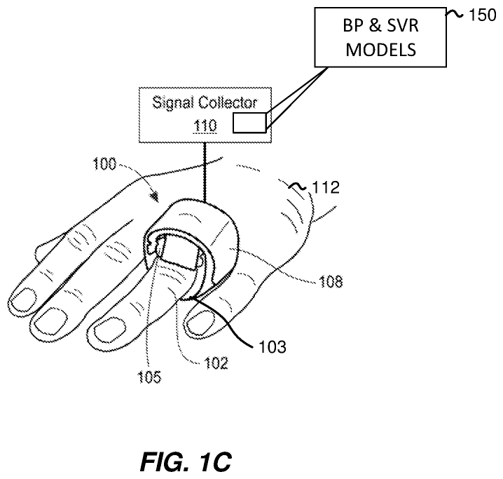

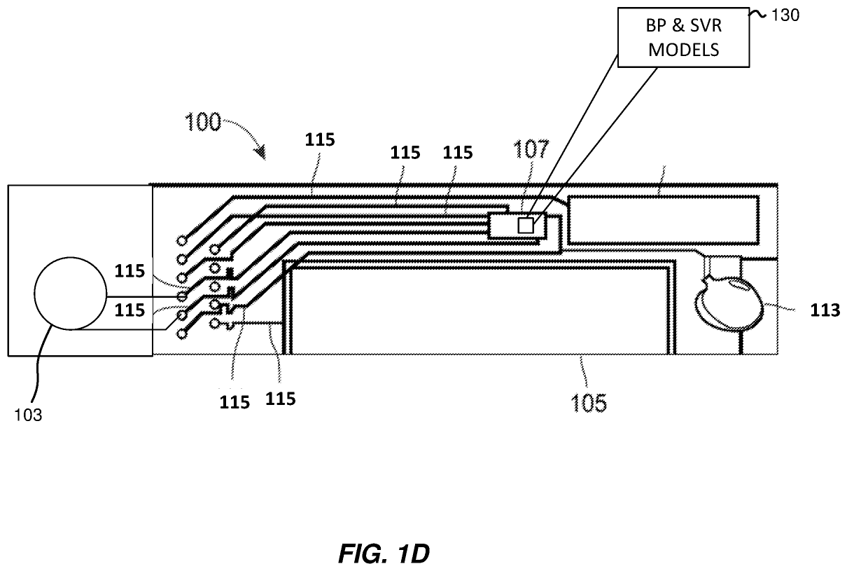

FIG. 1C illustrates an example sensor device 100 in the form of a multi-modal ring sensor 108 to be worn on a finger or digit 102 of a person or other subject, at a peripheral arterial region. FIG. 1C illustrates a distal limb portion of the subject, in this example a hand 112, where the peripheral arterial region resides. FIG. 1B illustrates another example sensor device 100′ similar to device 100 and showing a thin film structure that may be positioned as an inner dual sensor (or multi-modal) sensor structure of the device 100. FIG. 1A illustrates a proximal limb portion, in this example a forearm 114, at which is mounted a pressure cuff 116, in this example over the sensor device 100′, placing the device 100′ over a different example peripheral arterial region than that of the device 100. Albeit, in various other implementations, the pressure cuff 116 may be used in conjunction with the sensor device 100 mounted over the peripheral arterial region of FIG. 1C to affect the methods herein. That is, in some examples a sensor device may be positioned under the cuff. In some examples, the sensor device may be positioned externally away from the cuff. In FIG. 1A, the sensor device 100′ is positioned at a peripheral artery location of the subject, with example distal arteries 120 and 122 labeled. In some examples, the sensor device 100 is placed such that embedded sensors are positioned over peripheral arteries for tissue and sensor model development, for example. The sensor device 100/100′ may be positioned over any number of peripheral arterial regions including a finger, a wrist, an arm, a thigh, a calf, an ankle, a toe, a temple, a nose, a chest, and a neck, for example. In some implementations, the sensor device 100/100′ may be positioned around other artery accessible detection areas, including the neck or head of a subject. The example locations of the sensor device 100 and 100′ in FIG. 1C and FIG. 1B, respectively are example locations that may be used in a model development mode, in which data is collected on one or more subjects to develop a 2D model, or in an analysis mode, in which a developed model is used to assess hemodynamic variables indicated of a hemodynamic condition of a subject.

As shown in FIG. 1C, the sensor device 100 is employed in some implementations as a wearable sensor devices, which may be a ring sensor device. FIG. 1D is a diagram of an implementation of an electrical and mechanical layout of the sensor device 100 or sensor device 100′. The sensor device 100 includes a strain sensor 105, such as a piezoelectric sensor or other strain sensor, a photoplethysmogram (PPG) sensor 103, and a physical support structure in the form of a band 108 physically coupled to the strain sensor 105. The support structure may be any type of support for a sensor device that allows the sensors thereof to be operable in place on a subject for measuring respective signals. The support structure may be flexible or rigid, and in some examples removably affixable to a subject using a strap, Velcro, fastener, or other structure. The support structure may be coupled to the strain sensor 105 and the PPG sensor 103 to physically support the device 100 at a peripheral arterial region, for example. The sensor device 100′ includes the strain sensor 105 and PPG sensor 103, both of which operate in accord with that of sensor 100, therefore references herein to sensor 100 should be consider references to sensor 100′. The ring sensor further includes an electrical ground, a power source 113, circuitry 107, and electrical lead lines 115 for the strain sensor 105. The band 108 is configured to hold the strain sensor 105 in a position for the sensor to measure strain response of the subject (i.e., mechanical or other strain sensor, although in some examples the sensor may be a pulse sensor in some examples) and the PPG sensor 103 is positioned to measure blood volume changes in the microvasculature, e.g., arteries, at the peripheral region. The band 108 may include an elastic band, a Velcro band, a rubber band, medical tape, a buckle, strands for tying, or another band or material capable of holding the sensors 103 and 105 in a position. In some examples, the band 108 is a rigid or semi-rigid structure and the ring sensor device 100 is able to form an engaged fit around a finger that keeps the device in place for measurement. The ring sensor device 100 may include a tightening mechanism that allows for adjusting the pressure of the strain sensor 105 on the skin of the person. For example, a screw may be a tightening mechanism that allows a user to increase or decrease the pressure of the strain sensor 105 against the skin of the person. In another embodiment, a pressure/tension adjustment mechanism may be used that incorporates a bladder containing a gas or fluid, or a mechanical actuator such as those made of piezoelectric material.

The circuitry 107 may include an amplifier, low-pass filter, high-pass filter, bandpass filter, notch filter, amplifier, integrator, or another electrical element for performing signal conditioning and processing. Further, the circuitry 107 may include an integrated circuit, independent circuit elements (e.g., resistors, capacitors, operational amplifiers, etc.) an FPGA, or another circuit element. The ground creates a contact between skin of the person and acts as a grounding electrode which may be used to reduce noise due to the power source 113. In some implementations, the circuitry 107 includes hemodynamic models 130 stored therein, where those hemodynamic models 130 may include BP and SVR models, in accordance with the various methods described herein.

In implementations, the strain sensor 105 may be a PVDF sensor that does not need to be powered. A PVDF sensor can be a passive sensor that converts mechanical tension into an electrical current without requiring any external power supply. Therefore, in embodiments, the power source 113 may provide power to the circuitry 107. Further, the sensor device 100 may not include the circuitry 107, and any circuitry other than the strain sensor 105 may be in an external device and therefore, in embodiments, the sensor device 100 may also not include or require any power source. In embodiments, any circuitry for collecting and processing the signal from the strain sensor 105 may include a computer, tablet, phone (e.g., as performed by an app on a phone) server, cloud, mobile device, or other computational device.

In implementations, a signal collector 110 may be in communication with the sensor device 100. The signal collector 110 collects signals indicative of measured pulses from the ring sensor device 100. The signal collector 110 may perform further signal processing on the collected signals, and/or the signal collector 110 may provide the collected signals to another computer, or network for further processing of the signals. In embodiments, any or all of the circuitry 107 may be included in the signal collector 110. The signal collector 110 may be in communication with the sensor device 100 through wired or wireless communicative means. For example, the sensor device 100 may include an IO chip (e.g., having one or more processors and one or more memories) for providing communications with other computers, networks, and devices. The signal collector 110 may be a data acquisition system or a component of a data acquisition system. In embodiments, the signal collector 110 may collect the signals from the sensor device 100 and provide data indicative of the collected signals to a network. A person, at a remote location, may then use the disclosed methods to analyze the data to determine a BP, VR and/or CO of a person. For example, a user wearing the sensor device 100 may be at home, a grocery store, or driving in a car, and a medical professional stationed at a hospital may access data indicative of the pulses of the user. The medical professional can then determine, monitor, and/or log the BP, VR and/or CO of the person continuously in real time. In another embodiment, the data can be analyzed by the sensor or a stand-alone device or algorithm and stored for future use. For example, the extracted hemodynamic variables can be aggregated and used at a later date to assess the subject's health status. This information can be provided to the user through an application or website to give them feedback about their health.

The signal controller 110 in some implementations includes hemodynamic models 150 stored therein, where those hemodynamic models 150 may include BP and SVR models, in accordance with the various methods described herein.

### Example of Model Development Mode

In an example implementation, a sensor device, such as the ring sensor device 100 was operated in a model development mode executed using swine subjects to develop a hemodynamic model stored and executable in a controller, such as the controller 110.

While varying external pressure is a common aspect of non-invasive BP monitoring, external pressure modulation is not typically applied to photoplethysmograms. However, to develop a model for more sensor types, we hypothesized that trajectories of signals collected from multiple sensor modalities versus applied pressure can be used to refine estimates of hemodynamic quantities, such as changes in systemic vascular resistance and blood pressure.

Blood pressure and flow along arterial vessels (e.g., arteries 120 and 122) are affected by arterial dimensions and tissue properties. The piezoelectric sensor, as type of strain sensor, responds predominantly to tangential stress or strain in the PVDF film as it stretches in response to fluctuations in pressure and volume inside the underlying arteries. However, the pressure experienced by a PVDF ring sensor (e.g., the sensor 105 of the device 100) is not identical to the underlying arterial pressure, due to intervening tissue dynamics. Further, the voltage output of a PPG sensor (e.g. the sensor 103 of the device 100) is ideally proportional to the change of the artery's volume. However, due to the varying depth between the photodiode and artery when surrounding tissue is compressed, the illumination is also related to tissue dynamics. To approximate this behavior, a 2D model of local arterial and tissue geometry was developed to relate outside cuff pressure and internal artery properties to the signals from each of the different sensors.

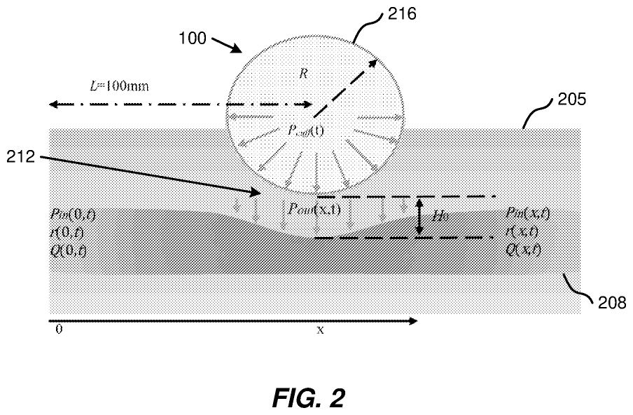

FIG. 2 illustrates a cross-section view of an example position of the sensor device 100 in the example, mounted to a swine fore-limb 205 that was separately under pressure via a pressure cuff 216, like the pressure cuff 116. The illustrated example was used in a model development mode of the sensor device.

For the illustrated example, the hemodynamic model developed was a mechanical model that included an artery 208 approximated as an elastic cylinder with linear elastic modulus, other soft tissue and skin approximated as a compressible volume; and the sensor device 100 approximated as a linear elastic cylindrical ring, as shown schematically in FIG. 2. The pressure cuff 216 was modeled as a transverse cylinder, and the artery 208 was modeled to a small distance upstream and downstream from the sensor location (100 mm in the current setting). In the example, the cuff 216 was wrapped around the appendage significantly past the artery 208 location on either side circumferentially.

The pressure outside the artery 208, Pₒᵤₜ, assuming an approximately cylindrical cuff geometry, was treated as following a single spatial period of a cosine curve along the length of the artery 208, with peak pressure equal to that inside the cuff 216. Under this model, Pₒᵤₜ as a function of position was written as:

$${P}_{out} ( x , t ) = \{ \begin{matrix}{P}_{cuff} ( t ) cos ( \frac{π \vert x - L \vert}{2 R} ) & , \vert x - L \vert \leq R \\ 0 & , \vert x - L \vert > R\end{matrix} \qquad ( 1 )$$

where x refers to linear distance along the artery 208, P_{cuff} is the pressure inside the cuff 216, R is a radius of the cuff 216, and L is the position of the midpoint of the cuff 216 along the x-axis.

Tissue 212 between the artery 208 and the cuff 216 was approximated by a piecewise-linear model: first, as a linear compressible volume under low to moderate pressures, then as having negligible compression (i.e., solid or rigid behavior) beyond a threshold pressure. Under this hypothesized behavior, distance between the PVDF sensor and the artery 208, H_{PVDF}, becomes:

$${H}_{PVDF} ( t ) = \{ \begin{matrix}{H}_{0} + k [ {P}_{0} - Δ P ( L , t ) ] & , Δ P ( L , t ) < {P}_{0} \\ {H}_{0} & , Δ P ( L , t ) \geq {P}_{0}\end{matrix} \qquad ( 2 )$$

$$Δ P ( L , t ) = {P}_{out} ( L , t ) - {P}_{in} ( L , t ) \qquad ( 3 )$$

where, H₀ is the minimum distance at which the tissue 212 acts as an incompressible solid, P₀ is the threshold pressure, and k is an effective spring constant for the tissue 212 in the compressible range. Pᵢₙ(x, t) is the blood pressure inside the artery 208 while ΔP is the difference between internal and external pressure on the artery wall. In this example, the PVDF sensor was taken to be centered underneath the cuff 216, and the PPG sensor was positioned 8 mm upstream, with light emission centered over the artery 208. More generally, placement of either or both of the sensors could be swapped or shifted relative to the cuff 216, with appropriate offset from L in functions for ΔP, Pₒᵤₜ, etc.

The distance between the PPG sensor and the artery 208 can be written similarly, adjusted just by the 8 mm offset:

$${H}_{PPG} ( t ) = \{ \begin{matrix}{H}_{0} + k [ {P}_{0} - Δ P ( L - 8 , t ) ] & , Δ P ( L - 8 , t ) < {P}_{0} \\ {H}_{0} & , Δ P ( L - 8 , t ) \geq {P}_{0}\end{matrix} \qquad ( 4 )$$

Longitudinally, the arterial vessel is modeled as an elastic cylindrical tube, shown in its lengthwise cross-section in FIG. 2. The viscous effects of blood and vessel viscoelasticity are neglected in the present model due to the comparatively short arterial distance examined; rather, emphasis is on distribution of radial artery deformation as a function of position near the sensors. The compliance, C, of the tube is defined as the change in tube cross-sectional area, A, divided by the change in pressure inside the tube, Pᵢₙ:

$$A ( x , t ) = π {r ( x , t )}^{2} \qquad ( 5 )$$

$$C ( x , t ) = dA / {dP}_{in} ( x , t ) \qquad ( 6 )$$

where r is the internal radius of the artery 208. From LaPlace's Law for a cylindrical tube, the compliance also can be determined by the geometry of the tube in addition to the elastic modulus of the tube wall:

$$C ( x , t ) = \frac{2 π {r ( x , t )}^{3}}{E ( x , t ) h} \qquad ( 7 )$$

where h is the wall thickness. E, the elastic modulus, increases with the difference between inside and outside pressure. For example, E has been shown to be related to Pₒᵤₜ and Pᵢₙ for arteries as follows:

$$E ( x , t ) = {E}_{0} {e}^{α ( {P}_{out} ( x , t ) - {P}_{in} ( x , t ) )} \qquad ( 8 )$$

where E₀ and α>0 are specific parameters for different individuals and arteries. E is assumed to only depend on pressure here.

By applying conservation of momentum, the tube can be modeled with the following equations:

$$Q ( x , t ) - Q ( x + dx , t ) + \frac{d ( A d x )}{d t} = 0 \qquad ( 9 a )$$

$$A [ {P}_{in} ( x , t ) - {P}_{in} ( x + dx , t ) ] = ρ \frac{d Q ( x , t )}{d t} dx \qquad ( 9 b )$$

where [symbol] is volume flow rate, and ρ is blood density.

Combining (6) and (9a), the tube model can be rewritten as:

$$\frac{d Q ( x , t )}{d x} + C ( x , t ) \frac{d {P}_{in} ( x , t )}{d t} = 0 \qquad ( 10 a )$$

$$A ( x , t ) \frac{d {P}_{in} ( x , t )}{d x} + ρ \frac{d Q ( x , t )}{d t} = 0 \qquad ( 10 b )$$

Combining (5) to (10), a nonlinear function relating artery radius to blood pressure and flow can be obtained as the following equations, discretized in time and space increments (dt and dx):

$$Q ( x , t ) - Q ( x + dx , t ) + C ( x , t ) [ {P}_{in} ( x , t ) - {P}_{in} ( x , t + d t ) ] = 0 \qquad ( 11 a )$$

$$Q ( x , t ) - Q ( x , t + dt ) + \frac{A ( x , t )}{ρ} [ {P}_{i n} ( x , t ) - {P}_{in} ( x + dx , t ) ] = 0 \qquad ( 11 b )$$

$$2 π r ( x , t ) [ r ( x , t ) - r ( x + dx , t ) ] = \frac{2 π {r ( x , t )}^{3}}{E ( x , t ) h} [ {P}_{in} ( x , t ) - {P}_{in} ( x + dx , t ) ] \qquad ( 11 c )$$

$$r ( 0 , t + dt ) = r ( dx , t ) . \qquad ( 11 d )$$

With known inputs of Pᵢₙ(0, t) and Q(0, t) from invasive cardiovascular catheterization, the discretized equations in (11) can be numerically solved given an initial value r(0,1). Here, the integration steps are chosen as dx=0.1 mm for space and dt=0.005 s for time, the latter the same as the experimental sampling time.

The resulting voltage outputs of the PPG and PVDF sensors, U_{PPG} and U_{PVDF}, respectively are modeled following as:

$${U}_{PPG} ( t ) = {K}_{ppg} {H}_{ppg} ( t ) \int {f}_{ppg} ( t - τ ) π {r ( L , τ )}^{2} {L}_{ppg} d τ \qquad ( 12 )$$

$${U}_{PVDF} ( t ) = {K}_{pvdf} \int {f}_{pvdf} ( t - τ ) {d}_{31} {E}_{1} {A}_{1} \{ 1 + \frac{[ 2 r ( L - 8 , τ ) + {H}_{pvdf} ( t ) ]}{{R}_{arm}} \} d τ \qquad ( 13 )$$

where K's are the gains of the respective sensors as denoted by subscripts _{PPG} and _{PVDF}, f's are the impulse responses of any electronic filters. L_{ppg} is the length of the segment of the artery 208 directly under the PPG sensor, assumed to be constant. E₁ is the elastic modulus of PVDF, A₁ is the surface area of the PVDF and d₃₁ is the PVDF piezoelectric strain coefficient. The total radius of the limb 205 is treated as a constant value, Rₐᵣₘ.

Using the above model expressed as Equations (12) and (13), anticipated voltage output, as a function of internal and external pressure, can be calculated once parameters such as R, H₀, etc. are identified.

### Experimental Data Collection

This section describes the experimental set-up and sample experimental data used to evaluate the model for arterial interaction with the wearable sensors.

Swine test subjects were anesthetized for cardiovascular testing, and maneuvers were performed in accordance with University of Michigan IACUC approved animal protocol PRO00006553. Arterial blood pressure (ABP), cardiac output (CO), volumetric flow, and central venous pressure (CVP) data were collected via carotid artery and pulmonary artery catheterization.

The PVDF strain sensor and a PPG sensor were placed on the foreleg of the subjects. The sensors were covered by an air-filled cuff, which controlled the surface pressure on the swine foreleg when supplied by an air pump and valve. The air pressure inside the cuff was measured by a 1.2 MPa pressure transducer. All signals were collected and stored using a BIOPAC MP150 data logging system and analyzed retrospectively.

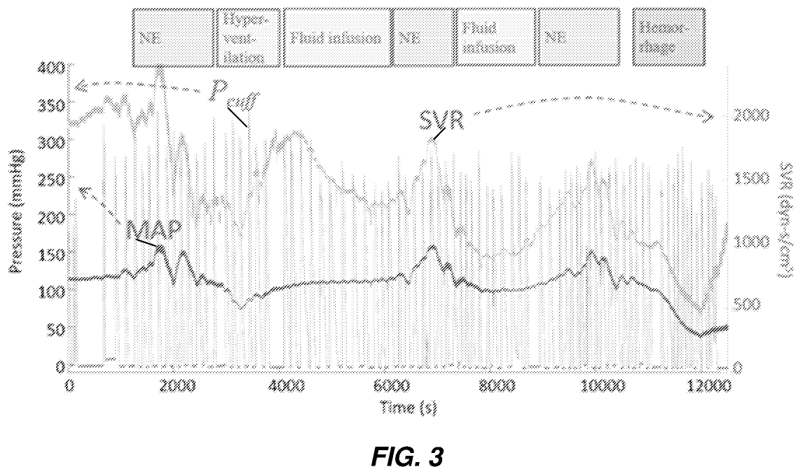

As shown in the experiment workflow in FIG. 3, changes in systemic vascular resistance (SVR) and ABP were induced by periodic intravenous infusion of the vasoconstrictor norepinephrine (NE), with SVR and mean arterial pressure increasing during infusion and decreasing when each infusion stopped. The infusion flow rate was controlled by an infusion pump, with a constant NE concentration (8 mcg/mL). Each animal underwent three rounds of NE infusions. In addition, the animals underwent hyperventilation to induce vasodilation. Fluid infusions were performed between maneuvers to allow for validation of the model at different levels of intravascular blood volume. In addition, the animals were hemorrhaged at the end of the experiment to simulate a low blood volume/low blood pressure state. Through these maneuvers, we were able to simulate the entire spectrum of hemodynamic parameters, i.e., MAP ranged from 40-160 mmHg (normal range: 70-105 mmHg) and SVR ranged from 500-2500 dyn·sec/cm⁵ (normal range: 800/1200 dyn·sec/cm⁵).

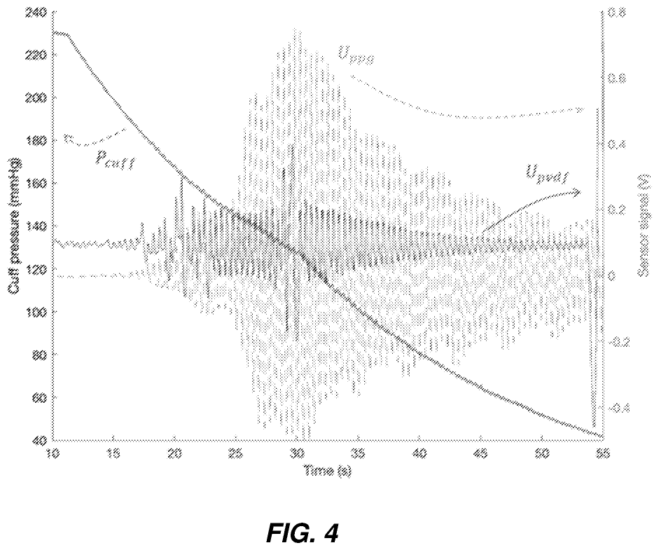

During these maneuvers, for each cuff pressure test the cuff was inflated to a maximum pressure of 380 mmHg, then air in the cuff was slowly released using a valve so that pressure dropped continuously to 0 mmHg over a period of approximate 1 minute, as shown in FIG. 4.

After completion of maneuvers for each swine, SVR was computed from cardiac output (CO), central venous pressure (CVP), and mean arterial pressure (MAP), expressed mathematically as SVR=(MAP−CVP)/CO. The experimental data analyzed in this example includes three swine subjects with nine NE infusion start/stop sequences and 137 cuff pressure tests in total. A sample trajectory for applied pressure, MAP, and SVR across a complete swine experiment is shown in FIG. 3. Indeed, it is noted that in some implementations, models are used to determine BP and/or SVR from measured output signals from the PVDF and PPG sensors. However, other hemodynamic variables may be obtained including CO, CVP, MEP, etc. and other described herein. In some examples, such as cardiac output, these variables may be determined from changes in the BP or SVR. In some examples, the models of BP and SVR may further include models for such variables, such as a model for CO.

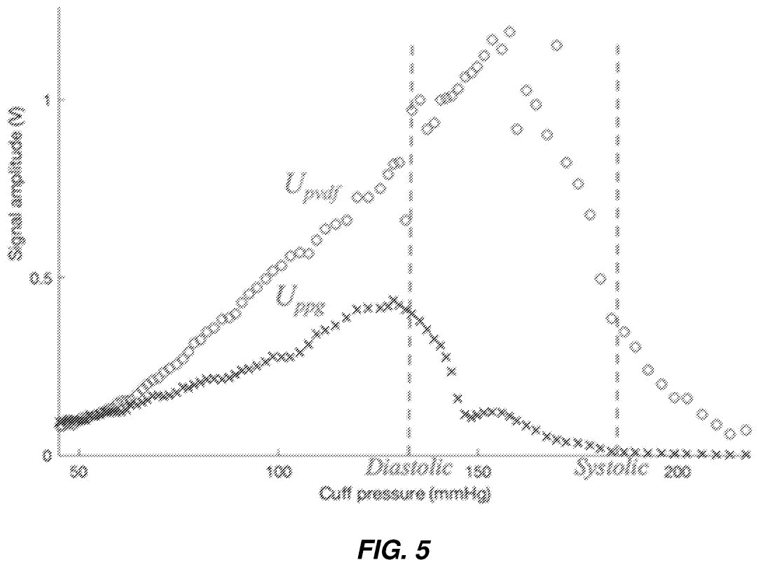

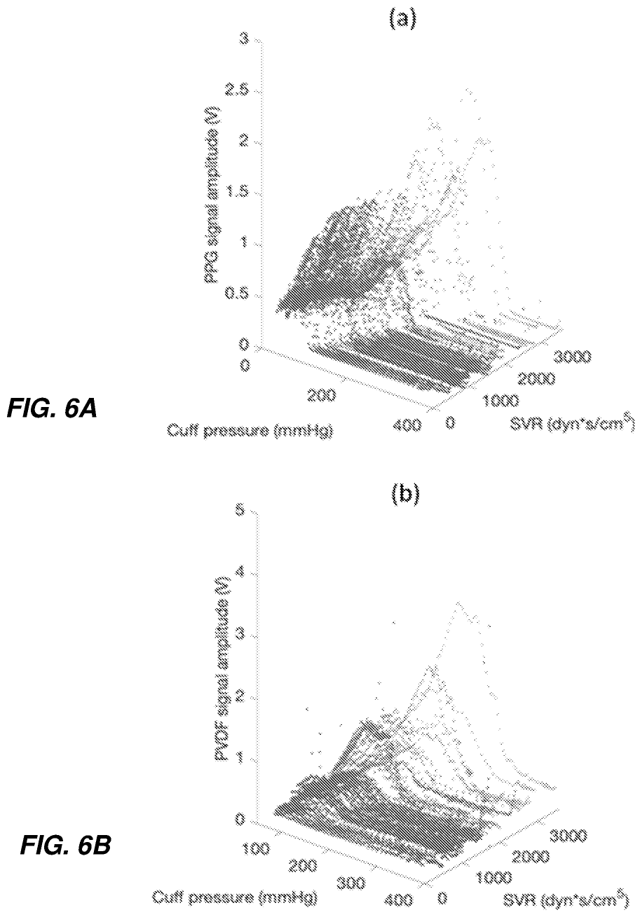

Qualitatively, for a given value of SVR at various points during the test, comparative PVDF sensor and PPG sensor trajectories were observed to have repeatable features. For example, FIG. 5 shows signal amplitude of PVDF and PPG sensor output signals versus applied cuff pressure for a single pressure test. However, when SVR and ABP are altered, the behavior of both PVDF and PPG sensor signals undergoes significant changes, especially in relationship to external cuff pressure. FIGS. 6A and 6B illustrate sample trajectories of PPG and PVDF sensor signals' heartbeat-to-heartbeat amplitudes against cuff pressure, organized from minimum to maximum SVR. Notable features of the sensors' responses to varying external cuff pressure include: complete elimination of PPG signal at high cuff pressure while a non-zero PVDF signal remains present; large differences in PVDF amplitude under high cuff pressure as SVR changes; and amplitudes that rise to a maximum then decrease for both signals as cuff pressure decreases, though maxima occur at very different times/pressures.

We further used the developed model to capture these prominent features of PVDF sensor and PPG sensor behaviors.

Parameter Identification: To evaluate whether the proposed tissue, artery, and sensor model can replicate the characteristic pressure- and SVR-dependent behaviors described above, parameters in the model were quantified using a combination of literature values and parameter identification. For many parameters, representative values from the literature were selected as approximate values and applied as constants, as listed in Table 1. Meanwhile, upon comparison of modeled deformation to experimental measurements, behavior associated with the values of the elastic modulus parameters E₀ and α in Equation (8) were observed to vary substantially among the swine subjects. As a result, these parameters were estimated individually by optimizing the fit between the measured sensor signals and the simulated signal. This identification was performed using the first 8 cuff pressure tests from each swine, to calibrate parameters E₀ and α.

SVR estimation with features from cuff deflation: As stated above, we hypothesized that features of the PVDF and PPG signal waveforms observed under cuff perturbation could be used to produce improved estimates of SVR compared to existing non-invasive measurement techniques. To test this hypothesis in the context of an individual swine, data from 50% (n=69) of the cuff inflation-deflation cycles was randomly selected as a training set for feature correlation with invasive SVR measurements. The remaining 50% (n=68) was used as a test set. In identification of an SVR model, different feature vectors were constructed from sensor amplitude versus cuff pressure profiles. These feature vectors usually have different dimensions. Therefore, the data was normalized to improve comparability among features. Normalization can also be effective in reducing adverse effects caused by outliers and speed up gradient descent to find an optimal solution from training data. In an example, a min-max normalization method was used to linearly transform the data to the range of [0, 1].

TABLE 1
Model parameters: representative tissue properties for nominal
model values taken from literature with exception of
nonlinear tissue elasticity parameters, identified from best
fit to first 8 cuff cycles for each swine.

| | | | |
|---|---|---|---|
| Variable | Parameter | Value | Units |

Nominal values used throughout testing

| r(0, 1) | initial artery radius | 1.5 | | | mm |
|---|---|---|---|---|---|
| Rₐᵣₘ | limb radius | 50 | | | mm |
| E₁ | sensor modulus | 2.5 | | | GPa |
| t | PVDF thickness | 52 | | | μm |
| d₃₁ | piezoelectric coefficient | 11 × 10⁻¹² | | | C/N |
| K_{ppg} | gain of PPG | 2000 | | | |
| K_{pvdf} | gain of PVDF | 200 | | | |
| ρ | blood density | 1060 | | | kg/m³ |
| L | location of cuff | 100 | | | mm |
| H₀ | initial radial sensor distance | 2 | | | mm |
| K | Tissue spring constant | 1.12 × 10⁻² | | | mm/mmHg |
| Identified from first 8 inflations, | | swine number | | | |
| used in further tests for each swine | | 1 | 2 | 3 | |
| E₀ | elastic modulus | 8.4 | 9.6 | 6.5 | kPa |
| α | rate of elasticity to blood | 15 | 12 | 10 | kPa/mmHg |
| | pressure | | | | |
| P₀ | tissue threshold pressure | 30 | 30 | 25 | mmHg |

TABLE 2
Linear regression features

| | | | Relative |
|---|---|---|---|
| | | | Contribution |
| Feature | Definition | Coefficients | Ratio (%) |
| P_{max, PVDF} | Cuff pressure when | 30.4 | 2.32 |
| | PVDF signal at | | |
| | maximum | | |
| P_{min, PVDF} | Cuff pressure when | −22.3 | 3.18 |
| | PVDF signal at minimum | | |
| P_{max, PPG} | Cuff pressure when PPG | 40.7 | 5.44 |
| | signal at maximum | | |
| P_{min, PPG} | Cuff pressure when PPG | −2.9 | 2.87 |
| | signal at minimum | | |
| V_{max, PVDF} | Maximum signal voltage | 595.9 | 3.35 |
| | of PVDF | | |
| V_{max, PPG} | Maximum signal voltage | 100.3 | 8.57 |
| | of PPG | | |
| k_{up, PPG} | Slope of PPG signal | −1.5 | 5.36 |
| | versus cuff pressure in | | |
| | rising | | |
| k_{down, PPG} | Slope of PPG signal | 14.7 | 6.35 |
| | versus cuff pressure in | | |
| | falling | | |
| k_{up, PVDF} | Slope of PVDF signal | 0.23 | 4.11 |
| | versus cuff pressure in | | |
| | rising | | |
| k_{down, PVDF} | Slope of PVDF signal | 0.07 | 2.12 |
| | versus cuff pressure in | | |
| | falling | | |
| R_{k, PPG} | k_{up, PPG}/k_{down, PPG} | −0.61 | 9.54 |
| R_{k, PVDF} | k_{up, PVDF}/k_{down, PVDF} | 445.0 | 12.27 |
| R_{V} | V_{max, PVDF}/V_{max, PPG} | −298.5 | 12.17 |
| Rᵤₚ | k_{up, PVDF}/k_{up, PPG} | 250.3 | 12.56 |
| R_{down} | k_{down, PVDF}/k_{down, PPG} | −40.7 | 7.78 |

Multiple linear regression (MLR) has been widely applied as a method to analyze the correlation, correlation direction, and strength between multiple independent variables and a dependent variable. Therefore, in example, an MLR model was developed for SVR, and the model parameters were estimated using the least squares method. The model was expressed as follows:

$$\hat{SVR} = {β}_{0} + {β}_{1} {x}_{1} + {β}_{2} {x}_{2} + … + {β}_{k} {x}_{k} , \qquad ( 14 )$$

where [symbol] is estimated systemic vascular resistance, β₁, β₂, . . . , βₖ are coefficients, and x₁, x₂, . . . , xₖ are input features.

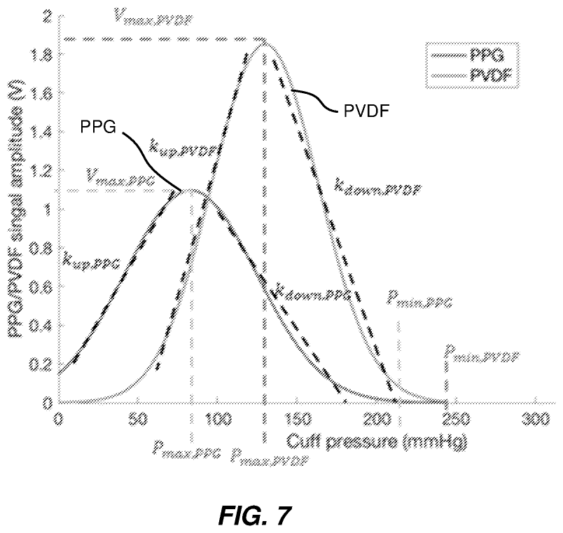

In an implementation, the input features for the SVR model were selected from the profile of PPG and PVDF signal amplitude versus cuff pressure, shown in FIG. 7 (showing line fit profiles) and summarized in Table 2. Key information from the features included the maxima and minima of the signal amplitudes, as well as the rate of increase and decrease of signal amplitudes as cuff pressure hanged. Impact on estimation of SVR is discussed in the following section. Relative contribution ratio (RCR) of candidate features was calculated by following function:

$$RCR ( {x}_{i} ) = \frac{{V}_{i}}{{\sum }_{i = 1}^{n} {V}_{i}} \qquad ( 15 )$$

where Vᵢ is total residual without xᵢ, and is tabulated in Table 2.

Blood Pressure Estimation with Model Refinement: A further hypothesis in our work is that using multiple sensors' information can produce more accurate BP estimates than oscillometric information alone. Oscillometric devices are widely used for automatic cuff BP measurement. Most of these devices estimate BP from the oscillometric cuff pressure waveform. For example, the standard method is to first estimate mean BP (mean pressure, MP) as the cuff pressure at which the oscillogram is maximal and then estimate each of systolic and diastolic BP (SBP and DBP) as the cuff pressure at which the oscillogram is some fixed ratio of its maximal value. The fixed ratio values are determined by obtaining the oscillogram and reference BP from a group of subjects and then finding the values that maximize the agreement between the estimated and reference BP in the group.

In the present example, both PPG and PVDF sensors' waveforms can be used to estimate SBP and DBP. An example method to estimate SBP and DBP is shown in Table 3. This method used an arbitrary value as a threshold to determine the time at which SBP and DBP are encountered during cuff inflation, instead of a fixed ratio of the maximal value. The time points are determined based on the observed sensor amplitude profiles versus cuff pressure, and thus do not require further calibration.

The refined algorithm of BP estimation combines the simple method and 2D artery model to determine SVR, as shown in the method of Table 4. The method of Table 3 gives an initial value and the boundary region of input pressures. The artery model is then used to predict PVDF and PPG sensors' voltage amplitude with different hypothesized BP input values. The estimated BP can then be found by minimizing the mean square root error between predicted signal amplitude and observed signal amplitude of the sensors for all cardiac cycles during the deflation of the cuff.

TABLE 3
BP estimation based on signal amplitude of two sensors

1. Deflate cuff pressure from 400 mmHg to 0 mmHg
2. Record the voltage signal amplitude of PVDF and PPG
sensors for each cardiac cycle, denoted V_{PVDF, i} and V_{PPG, i},
where i is the number of cardiac cycles.
3. Identify pressure when PPG signal reappears: If
V_{PPG, i} > 0.2, then P̅_{sys} = P_{cuff, i}, where P̅_{sys} is the
estimated systolic BP, P_{cuff, i} is the mean of cuff
pressure in number i-th th cardiac cycle.
4. Identify the pressure at which PVDF signal is maximized:
If V_{PVDF, i} = maximum {V_{PVDF, n}, n = 1, 2, . . . N},
then P̅_{dia} = P_{cuff, i}, where P̅_{dia} is the estimated diastolic
BP, N is the total number of cardiac cycles.

TABLE 4
BP estimation with 2D model correction

1. Repeat simple algorithm (Table 3) to get P̅_{sys}, P̅_{dia}.
2. Initate simulated voltage output from model: Set the initial profile
for Pᵢₙ(0, t) to have the same waveform shape as the PVDF signal
with maximum value of P̅_{sys}, and minimum value of P̅_{dia}.
3. Initiate hypothesized SVR for model simulation: Let Q(0, t) =
Pᵢₙ(0, t)/2000, 2000 is the standard SVR for swine test.
4. Simulate model response given hypothesized pressure range
and SVR: Numerically solved the discrete equation (11) with
inputs of Pᵢₙ (0, t) and Q(0, t).
5. Simulated modeled voltage amplitudes: Find simulated peak-to-
peak PVDF and PPG signal amplitudes, denoted V̅_{PPVDF,i} and V̅_{PPG,i},
as functions of applied cuff pressure from the model's output
of U_{PVDF}(t) and U_{PPG}(t).
6. Record root-mean-square cost function, J, from the current
simulation:
$J ( {P}_{in} ( 0 , t ) ) = \frac{1}{N} {\sum }_{i = 1}^{N} \sqrt{{( {\overline{V}}_{PVDF , i} - {V}_{PVDF , i} )}^{2} + {( {\overline{V}}_{PPG , i} - {V}_{PPPG , i} )}^{2}} ,$
subject to Pᵢₙ(0, t) ∈ {0.8P̅_{dia}, 1.2P̅_{sys}}.
7. Repeat steps 2-6 while adapting the hypothesized range of Pᵢₙ(0, t)
and SVR; Record refined pressure estimates, denoted \hat{P}_{dia} and
\hat{P}_{sys}, minimizing J.

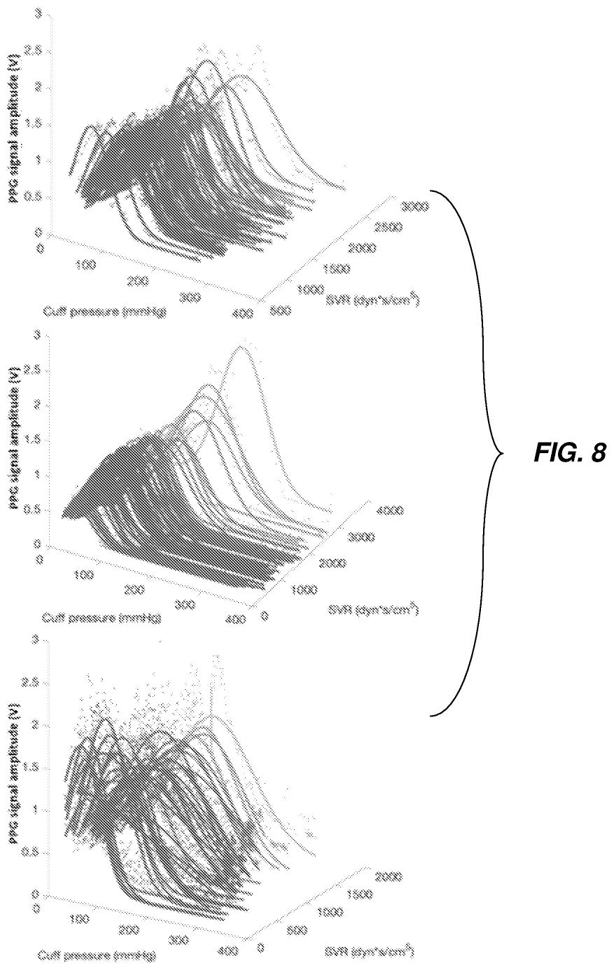

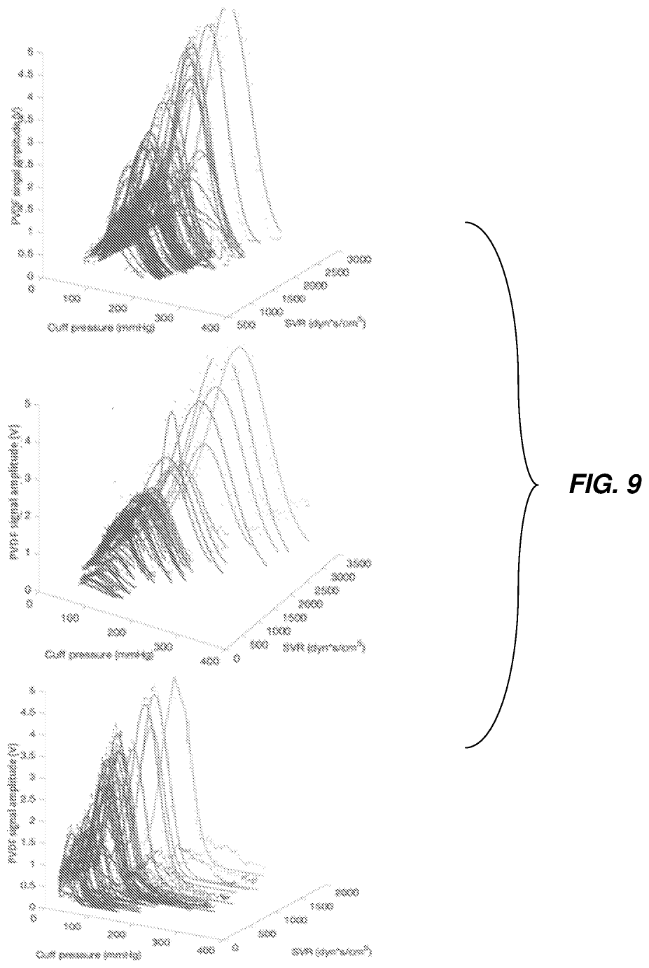

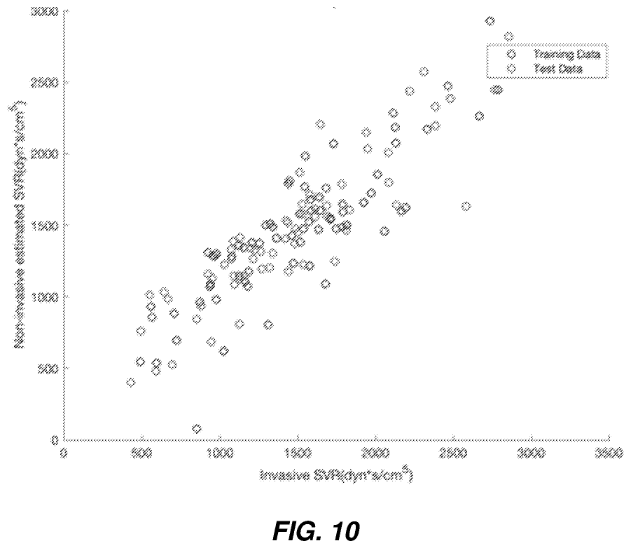

Results for modeled PVDF and PPG signal amplitudes as a function of mean SVR and applied cuff pressure after parameter identification are shown in FIGS. 8 and 9, respectively for three swine subjects in an example. Over the duration 137 measurements from the three experiments, diastolic pressure (DBP) varied from 53 to 119 mmHg and systolic pressure (SBP) varied from 73 to 220 mmHg, in response to maneuvers. Once elasticity parameters E₀ and α are identified for each subject, the proposed model is even more effective in predicting the signals' response in the same animal in future cuff inflation/deflation cycles. Importantly, significant changes in the arterial dynamics and the sensors signal amplitudes are observed with varying SVR, in the form of features such as the cuff pressures at which signal amplitudes are maximized and the amplitude at those maxima, suggesting that the model provides reasonable explanation of changes in wearable sensor behavior for varying SVR and applied external pressure.

For example, the peak amplitude of the PPG signal is observed when cuff pressure is below diastolic pressure, which can be explained by tissue dynamics in the model, as the maximum combination of arterial expansion and transmissibility of light (based on tissue thickness) occurs at that point. Meanwhile, the PPG signal is close to zero when cuff pressure is higher than systolic pressure, as would be expected given suppression of artery expansion under high applied pressure. In contrast, the PVDF signal amplitude reaches its maximum when cuff pressure is very close to diastolic pressure; this is consistent with maintaining a large proportional change in strain at the PVDF sensor when accounting for compression of the tissue. At high cuff pressures, presence of a measurable PVDF signal when PPG signal has been eliminated is additionally consistent with longitudinal effects of pressure propagation incorporated in the model.

Results from FIGS. 8 and 9 are consistent with previously proposed methods for tracking changes in vascular resistance, which found that variations in relative amplitude and hysteresis between the PVDF and PPG signals show strong correlations with invasively measured SVR. Specifically, when applied pressure is modestly below diastolic pressure, both sensors operate near their peak amplitudes, and correlation coefficient was higher than that for SVR estimation from PPG or PPG and PVDF sensing in previous studies.

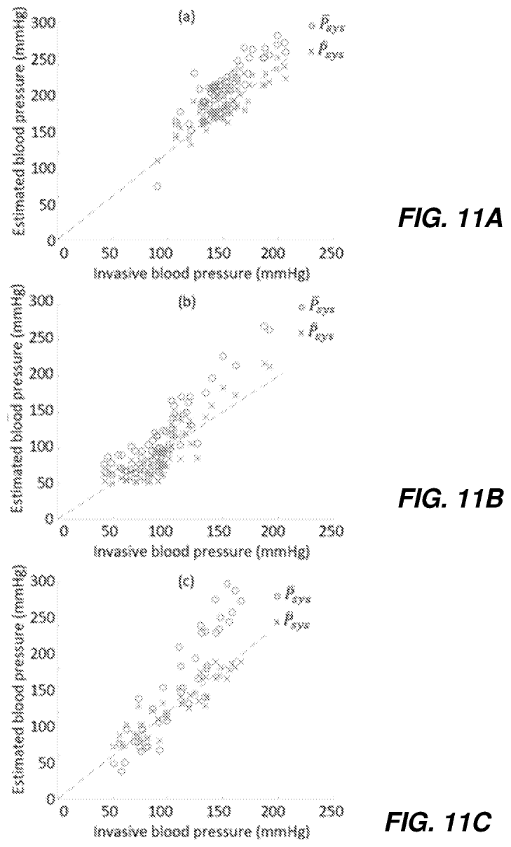

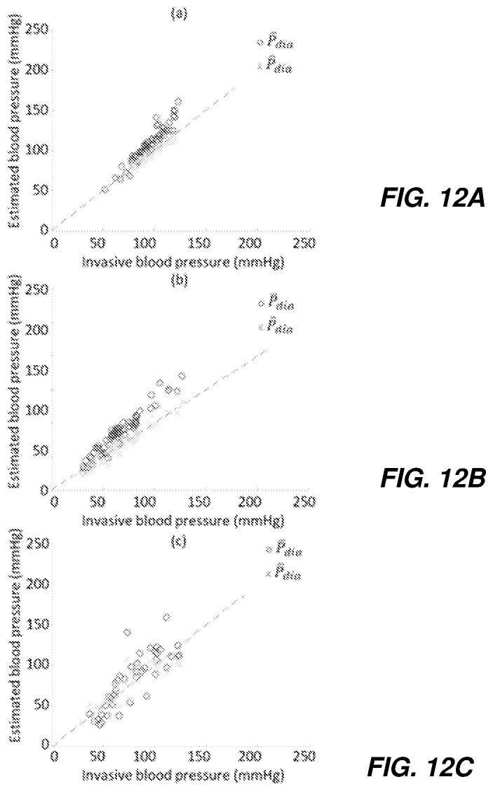

This work further allows refinement of non-invasive blood pressure measurements. FIGS. 11A-11C and 12A-12C summarize, respectively, the SBP and DBP estimation accuracy using the proposed initial estimation method (Table 1, features directly from amplitude versus cuff pressure trajectory) and the refined method with the 2D model (Table 2, best fit of modeled voltage outputs over full cuff deflation, for hypothesized SBP and SVR). Estimated blood pressure is validated against invasive measurements. These results were obtained from three swine subjects with 137 measurements. RMSE of the simple estimation method ranged from 13.3 to 42.2 mmHg and 7.6 to 36.0 mmHg for SP and DP, respectively. The refined algorithm using the full 2-D model had significantly lower RMSE, from 9.8 to 12.9 mmHg and 4.1 to 10.6 mmHg for SBP and DBP, respectively. Hence, the model-refined method was able to maintain the high accuracy over a wide BP range. Furthermore, the precision errors of this method were significantly lower relative to the simple method and patient-specific methods described in prior literature. We also note that the errors for high SBP cases using the simple method were much higher than those for low SBP cases. This could be due to the high stiffness of the artery when undergoing peripheral vessel constriction; this would be consistent with the model-refined method correcting for this nonlinearity by introducing the pressure-related elastic modules of artery wall in (8).

As shown, the techniques herein may include a model for local longitudinal and radial artery motion under a compliant wearable sensor to explain changes in amplitude of multi-modal sensors (PPG and piezoelectric PVDF) as exterior cuff pressure is manipulated. The model is highly effective in capturing characteristic amplitude profiles of both sensors when tested on swine subjects undergoing maneuvers to manipulate SVR and BP. The model requires modest calibration, primarily to identify subject-specific parameters for nonlinear tissue compressibility between the artery and non-invasive sensors.

There are many applications of the model to physiological monitoring. First, a regression model for SVR using features from sensors' amplitude versus external cuff pressure trajectories was obtained. The model showed consistent correlation across changes in SVR among the three swine subjects. Second, two methods for estimating systolic and diastolic BP from the two sensing modalities is presented, for comparison to traditional oscillometric measurements. High accuracy BP estimates were obtained by identifying hypothesized blood pressure amplitudes that best fit simulated sensor voltage outputs to observed voltage outputs. Thus, the techniques indicate that comparing measurements of peripheral arterial motion using multiple sensor modalities can refine information on cardiovascular function when performing cuff-based, non-invasive cardiovascular monitoring.

TABLE 5
Root-mean-square-error (RMSE) for systolic and diastolic
pressures measured by simple (Table 3) and refined
(Table 4) algorithms for estimating BP using PPG and
PVDF sensors.
RMSE (mmHg)

| | P̅_{sys} | \hat{P}_{sys} | P̅_{dia} | \hat{P}_{dia} |
|---|---|---|---|---|
| Swine 1 | 13.2786 | 9.8581 | 7.6040 | 4.1252 |
| Swine 2 | 23.7746 | 11.1469 | 8.5588 | 6.6068 |
| Swine 3 | 42.2365 | 12.8665 | 35.9417 | 10.6520 |

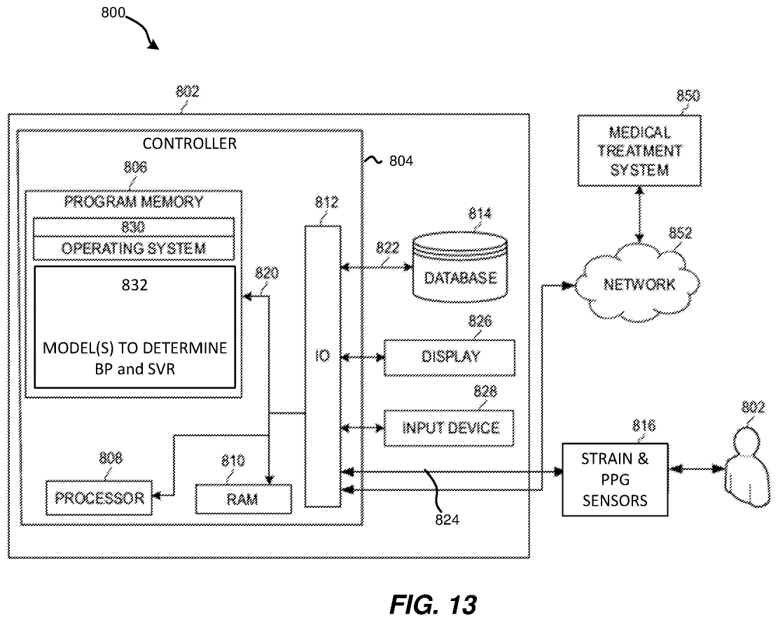

FIG. 13 is an example block diagram of an example hemodynamic measurement system 800, illustrating the various components used in implementing an example embodiment of processes described herein. A signal-processing device 802 (or “signal processor”) may be configured to examine a patient or healthy subject 820 via one or more sensors of a sensor device 816 in accordance with executing the functions of the disclosed embodiments. The sensor device 816 may be one or more wearable sensors such as the ring sensor device 100, for example, and include multiple sensors, including a strain sensor (e.g., PVDF sensor) and as sensor to measure blood volume changes in the microvasculature (e.g., a PPG sensor). The sensor device 816 may include any sensor capable of capturing mechanical strain responses in accordance with the methods herein (which would al, whether wearable or otherwise attachable to a subject.

The signal-processing device 802 may have a controller 804 operatively connected to a database 814 via a link 822 connected to an input/output (I/O) circuit 812. It should be noted that, while not shown, additional databases may be linked to the controller 804 in a known manner. The controller 804 includes a computer-readable media such as program memory 806, one or more processors 808 (may be called microcontrollers or a microprocessors), a random-access memory (RAM) 810, and the input/output (I/O) circuit 812, all of which are interconnected via an address/data bus 820. It should be appreciated that although only one processor 808 is shown, the controller 804 may include multiple microprocessors 808. Similarly, the memory of the controller 804 may include multiple RAMs 810 and multiple program memories 806. Although the I/O circuit 812 is shown as a single block, it should be appreciated that the I/O circuit 812 may include a number of different types of I/O circuits. The RAM(s) 810 and the program memories 806 may be implemented as semiconductor memories, magnetically readable memories, and/or optically readable memories, for example.

The computer-readable media 806 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units 808 of the computing device 802 may represent a CPU-type processing unit, a GPU-type processing unit, a field-programmable gate array (FPGA), another class of digital signal processor (DSP), or other hardware logic components that can be driven by a CPU.

A link 824, which may include one or more wired and/or wireless (Bluetooth, WLAN, etc.) connections, may operatively connect the controller 804 to the sensor(s) of the sensor device 816 through the I/O circuit 812. As a wearable sensor, the sensor device 816 may be operatively connected to the person 820 at a location corresponding to a sampling region of the person in accordance with techniques herein, including but not limited to a person's wrist, finger, arm, ankle, waist, leg, or another part of the body for measuring the pulse of the person.

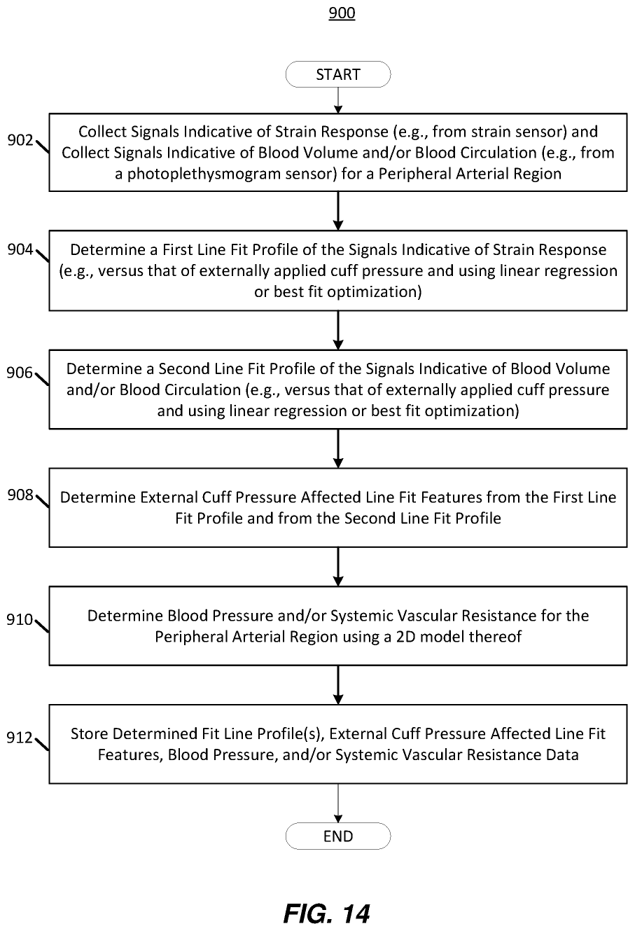

The program memory 806 and/or the RAM 810 may store various applications (i.e., machine readable instructions) for execution by the processor 808. For example, an operating system 830 may generally control the operation of the signal-processing device 802 and provide a user interface to the signal-processing device 802 to implement the processes described herein. The program memory 806 and/or the RAM 810 may also store a variety of subroutines 832 for accessing specific functions of the signal-processing device 802. By way of example, and without limitation, the subroutines 832 may include, among other things: a subroutine for determining the BP and/or SVR of a subject by applying one or more of the example models described. The subroutines 832 may implement any of the example methods described and illustrated herein to determine hemodynamic variables such as BP and/or SVR from captured sensor signals from a multi-mode sensor device implemented as sensors of the sensor device 816, including the methods described and illustrated in FIG. 14. These subroutines may be executed through one or more apps executed on the signal-processing device 802. The one or more apps may include a GUI interface for interaction by a user via the display screen 826. In the illustrated example, the subroutines 832 include performing pulse deconstruction, determining an incident and reflected wave, determining a reflection coefficient, and determining hemodynamic variables of the subject 820.

The subroutines 832 may also include other subroutines, for example, implementing software keyboard functionality, haptic touchscreen functionality, and/or interfacing with other hardware in the signal-processing device 802, etc. The program memory 806 and/or the RAM 810 may further store data related to the configuration and/or operation of the signal-processing device 802, and/or related to the operation of the one or more subroutines 832. For example, sensor data such as a pulse of a person may be data gathered by the sensor device 816, data determined and/or calculated by the processor 808, etc. In addition to the controller 804, the signal-processing device 802 may include other hardware resources. The signal-processing device 802 may also include various types of input/output hardware such as a visual display 826 and input device(s) 828 (e.g., keypad, keyboard, etc.). In an embodiment, the display 826 is touch-sensitive and may cooperate with a software keyboard routine as one of the software routines 832 to accept user input.

It may be advantageous for the signal-processing device 802 to communicate with a broader medical treatment system 850 through a network 852, using any of a number of known networking devices and techniques (e.g., through a computer network such as a hospital or clinic intranet, the Internet, etc.). For example, the system may be connected to a medical records database, hospital management processing system, health care professional terminals (e.g., doctor stations, nurse stations), person monitoring systems, automated drug delivery systems such as smart pumps, smart infusion systems, automated drug delivery systems, etc. Accordingly, the disclosed embodiments may be used as part of an automated closed loop system or as part of a decision assist system.

The network 852 may be a public network such as the Internet, private network such as research institution s or corporation s private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 852 can include a number of devices that facilitate network communications and/or form a hardware basis for the networks, such as switches, routers, gateways, access points (such as a wireless access point as shown), firewalls, base stations, repeaters, backbone devices, etc.

FIG. 14 illustrates an example method 900 non-invasively measuring one or more hemodynamic variables of a subject as may be performed by the system 800, in particular, at the signal-processing device 802. At a block 902, the signal-processing device (e.g., device 802) collects collect signals indicative of strain response (e.g., from a strain sensor of sensors 816) and collect signals indicative of blood volume and/or blood circulation (e.g., from a photoplethysmogram sensor of sensors 816) for a peripheral arterial region of the subject. at a block 904, the method 900 determine a first line fit profile of the signals indicative of strain response and, at block 906, the method 900 determine a second line fit profile of the signals indicative of blood volume and/or blood circulation. Example line fit profiles are described and illustrated in various examples herein. For example, the first line profile and/or the second line fit profile may be determined using linear regression or best fit optimizations, each different corresponding to the different collected signals. The block 904, for example, may determine the first line fit profile of the signals indicative of the strain response versus externally applied cuff pressure by comparing the signals indicative of the strain response versus externally applied cuff pressure against a previously measured strain response signal curve and performing an error minimization until the first line fit profile corresponds to the measure strain response signal curve. Similarly, the block 906 may determine the second line fit profile of the signals indicative of the blood volume and/or blood circulation changes versus externally applied cuff pressure by comparing the signals indicative of the blood volume and/or blood circulation changes versus externally applied cuff pressure against a previously measured photoplethysmogram response signal curve and performing an error minimization until the second line fit profile corresponds to the measured photoplethysmogram response signal curve.

At a block 908, the method 900 determines external cuff pressure affected line fit features from the first line fit profile of block 904 and the second line fit profile of block 906. Examples fit features determined at the block 908 include, by way of example, the linear regression features listed in Table 2, some of which are shown in FIG. 7, which illustrates example first and second fit line profiles.

At a block 910, the method 900 determines blood pressure and/or systemic vascular resistance for the peripheral arterial region using a 2D model, for example, expressed by Equations (12) and (13) and as discussed in examples above. The method 900 stores determined values (e.g., first line fit profile, second line fit profile, blood pressure, systemic vascular resistance data, external cuff pressure affected line fit features, etc.) at a block 912.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion such as a Contrast Agent Injection System shown in FIG. 3) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed is:

1. A device for non-invasively measuring one or more hemodynamic variables of a subject, the device comprising:
a strain sensor positioned in the device to generate signals indicative of strain response at a peripheral arterial region of the subject;
a photoplethysmogram sensor positioned in the device to generate signals indicative of a blood volume and/or blood circulation changes at the peripheral arterial region;
a support structure physically coupled to the strain sensor and the photoplethysmogram sensor to physically support the device at the peripheral arterial region; and
a processor in communication with the strain sensor and the photoplethysmogram sensor and configured to:
collect the signals indicative of the strain response and the signals indicative of the blood volume and/or blood circulation changes during changes in an externally applied cuff pressure to the subject;
determine a first line fit profile of the signals indicative of the strain response versus externally applied cuff pressure;
determine a second line fit profile of the signals indicative of the blood volume and/or blood circulation changes versus externally applied cuff pressure; and
determine external cuff pressure affected line fit features from the first line fit and from the second line fit and from the external cuff pressure affected line fit features determining a blood pressure and/or a systemic vascular resistance for the peripheral arterial region, as the one or more hemodynamic variables.

2. The device of claim 1, the processor is configured to:
determine the first line fit profile of the signals indicative of the strain response versus externally applied cuff pressure using a linear regression.

3. The device of claim 1, the processor is configured to:
determine the first line fit profile of the signals indicative of the strain response versus externally applied cuff pressure using a best fit optimization against a predetermined baseline fit profile.

4. The device of claim 1, the processor is configured to:
determine the second line fit profile of the signals indicative of the blood volume and/or blood circulation changes versus externally applied cuff pressure using a linear regression.

5. The device of claim 1, the processor is configured to:
determine the second line fit profile of the signals indicative of the blood volume and/or blood circulation changes versus externally applied cuff pressure using a best fit optimization against a predetermined baseline fit profile.

6. The device of claim 1, the processor is configured to:
determine the first line fit profile of the signals indicative of the strain response versus externally applied cuff pressure by comparing the signals indicative of the strain response versus externally applied cuff pressure against a previously measured strain response signal curve and performing an error minimization until the first line fit profile corresponds to the measure strain response signal curve.

7. The device of claim 1, the processor is configured to:
determine the second line fit profile of the signals indicative of the blood volume and/or blood circulation changes versus externally applied cuff pressure by comparing the signals indicative of the blood volume and/or blood circulation changes versus externally applied cuff pressure against a previously measured photoplethysmogram response signal curve and performing an error minimization until the second line fit profile corresponds to the measured photoplethysmogram response signal curve.

8. The device of claim 1, wherein external cuff pressure affected line fit features are comprise one or more features selected from:

| Feature | Definition |
|---|---|
| P_{max, PVDF} | Cuff pressure when strain sensor signal at maximum |
| P_{min, PVDF} | Cuff pressure when strain sensor signal at minimum |
| P_{max, PPG} | Cuff pressure when PPG sensor signal at maximum |
| P_{min, PPG} | Cuff pressure when PPG sensor signal at minimum |
| V_{max, PVDF} | Maximum signal voltage of strain sensor |
| V_{max, PPG} | Maximum signal voltage of PPG sensor |
| k_{up, PPG} | Slope of PPG signal versus cuff pressure in rising |
| k_{down, PPG} | Slope of PPG sensor signal versus cuff pressure in falling |
| k_{up, PVDF} | Slope of strain sensor signal versus cuff pressure in rising |
| k_{down, PVDF} | Slope of strain sensor signal versus cuff pressure in falling |
| R_{k, PPG} | k_{up, PPG}/k_{down, PPG} |
| R_{k, PVDF} | k_{up, PVDF}/k_{down, PVDF} |
| R_{V} | V_{max, PVDF}/V_{max, PPG} |
| Rᵤₚ | k_{up, PVDF}/k_{up, PPG} |
| R_{down} | k_{down, PVDF}/k_{down, PPG} |

9. The device of claim 1, the processor is configured to determine the blood pressure and/or the systemic vascular resistance for the peripheral arterial region using a 2D model of peripheral arterial region, the 2D model comprising an expression for pressure as a function of longitudinal length and cross-sectional radius inside the period arterial region.

10. The device of claim 9, wherein 2D model comprises the expressions:
$${U}_{PPG} ( t ) = {K}_{ppg} {H}_{ppg} ( t ) \int {f}_{ppg} ( t - τ ) π {r ( L , τ )}^{2} {L}_{ppg} d τ \qquad ( 1 )$$
$${U}_{PVDF} ( t ) = {K}_{pvdf} \int {f}_{pvdf} ( t - τ ) {d}_{31} {E}_{1} {A}_{1} \{ 1 + \frac{[ 2 r ( L - 8 , τ ) + {H}_{pvdf} ( t ) ]}{{R}_{arm}} \} d τ \qquad ( 2 )$$
where K's are the gains of the respective sensors as denoted by subscripts PPG and PVDF, f's are the impulse responses of any electronic filters, L_{ppg} is the length of the peripheral arterial region under the PPG sensor, assumed to be constant, E₁ is the elastic modulus of a PVDF material of the strain sensor, A₁ is the surface area of the PVDF material of the strain sensor and d₃₁ is the PVDF piezoelectric strain coefficient of the strain sensor, and the total radius of a location of an external cuff pressure is a constant value, Rₐᵣₘ, and t is time.

11. The device of claim 1, wherein the strain sensor is a piezoelectric sensor.

12. The device of claim 1, wherein the strain sensor is a polyvinylidene fluoride sensor.

13. The device of claim 1, wherein the processor is further configured to determine components of the systemic vascular resistance.

14. The device of claim 1, wherein the strain sensor is selected from the group consisting of: an optical sensor, a force based sensor, an electrical based sensor, and an ultrasonic sensor.

15. The device of claim 1, wherein the support structure is a band.

16. The device of claim 10, wherein the band configured to attach to a finger of the subject.

17. The device of claim 1, wherein the strain sensor is configured to sense a strain from a body part selected from the group consisting of: a finger, a wrist, an arm, a thigh, a calf, an ankle, a toe, a temple, a nose, a chest, and a neck.

18. The device of claim 1, further comprising an external cuff configured to apply the externally applied cuff pressure to the subject, wherein the external cuff is communicatively coupled to the process for providing externally applied cuff pressure values to the processor.

19. A method for noninvasive measurement of one or more hemodynamic variables of a subject, the method comprising:
using a strain sensor, collecting signals indicative of strain response at a peripheral arterial region of the subject during changes in an externally applied cuff pressure to the subject;
using a photoplethysmogram sensor, collecting signals indicative of a blood volume and/or blood circulation changes at the peripheral arterial region during the changes in the externally applied cuff pressure to the subject;
determining, in a processor, a first line fit profile of the signals indicative of the strain response versus externally applied cuff pressure;
determine, in the processor, a second line fit profile of the signals indicative of the blood volume and/or blood circulation changes versus externally applied cuff pressure; and
determine, in the processor, external cuff pressure affected line fit features from the first line fit and from the second line fit and form the external cuff pressure affected line fit features determining a blood pressure and/or a systemic vascular resistance for the peripheral arterial region, as the one or more hemodynamic variables.

20. The method of claim 19, further comprising determining the first line fit profile of the signals indicative of the strain response versus externally applied cuff pressure using a linear regression.

21. The method of claim 19, further comprising determining the first line fit profile of the signals indicative of the strain response versus externally applied cuff pressure using a best fit optimization against a predetermined baseline fit profile.

22. The method of claim 19, further comprising determining the second line fit profile of the signals indicative of the blood volume and/or blood circulation changes versus externally applied cuff pressure using a linear regression.

23. The method of claim 19, further comprising determining the second line fit profile of the signals indicative of the blood volume and/or blood circulation changes versus externally applied cuff pressure using a best fit optimization against a predetermined baseline fit profile.

24. The method of claim 19, further comprising determining the first line fit profile of the signals indicative of the strain response versus externally applied cuff pressure by comparing the signals indicative of the strain response versus externally applied cuff pressure against a previously measured strain response signal curve and performing an error minimization until the first line fit profile corresponds to the measure strain response signal curve.

25. The method of claim 19, further comprising determining the second line fit profile of the signals indicative of the blood volume and/or blood circulation changes versus externally applied cuff pressure by comparing the signals indicative of the blood volume and/or blood circulation changes versus externally applied cuff pressure against a previously measured photoplethysmogram response signal curve and performing an error minimization until the second line fit profile corresponds to the measured photoplethysmogram response signal curve.

26. The method of claim 19, wherein external cuff pressure affected line fit features are comprise one or more features selected from:

| Feature | Definition |
|---|---|
| P_{max, PVDF} | Cuff pressure when strain sensor signal at maximum |
| P_{min, PVDF} | Cuff pressure when strain sensor signal at minimum |
| P_{max, PPG} | Cuff pressure when PPG sensor signal at maximum |
| P_{min, PPG} | Cuff pressure when PPG sensor signal at minimum |
| V_{max, PVDF} | Maximum signal voltage of strain sensor |
| V_{max, PPG} | Maximum signal voltage of PPG sensor |
| k_{up, PPG} | Slope of PPG signal versus cuff pressure in rising |
| k_{down, PPG} | Slope of PPG sensor signal versus cuff pressure in falling |
| k_{up, PVDF} | Slope of strain sensor signal versus cuff pressure in rising |
| k_{down, PVDF} | Slope of strain sensor signal versus cuff pressure in falling |
| R_{k, PPG} | k_{up, PPG}/k_{down, PPG} |
| R_{k, PVDF} | k_{up, PVDF}/k_{down, PVDF} |
| R_{V} | V_{max, PVDF}/V_{max, PPG} |
| Rᵤₚ | k_{up, PVDF}/k_{up, PPG} |
| R_{down} | k_{down, PVDF}/k_{down, PPG} |

27. The method of claim 19, further comprising determining the blood pressure and/or the systemic vascular resistance for the peripheral arterial region using a 2D model of peripheral arterial region, the 2D model comprising an expression for pressure as a function of longitudinal length and cross-sectional radius inside the period arterial region.

28. The method of claim 19, wherein the strain sensor is a piezoelectric sensor.

29. The method of claim 19, wherein the strain sensor is a polyvinylidene fluoride sensor.
